(19) United States
(12) Patent Application Publication
(10) Pub. No.: US 2025/0313812 A1
(43) Pub. Date: Oct. 9, 2025

(54) METHOD FOR VIRUS CAPTURE

(71) Applicant: CYTIVA BIOPROCESS R&D AB, Uppsala (SE)

(72) Inventors: Jean-Luc Maloisel, Uppsala (SE); Ola Lind, Uppsala (SE)

(21) Appl. No.: 18/577,446

(22) PCT Filed: Jun. 21, 2022
(86) PCT No.: PCT/EP2022/066849

§ 371 Date: Jan. 8, 2024

(30) Foreign Application Priority Data
Jul. 8, 2021 (GB) 2109864.5

(51) Int. Cl. C12N 7/00

(52) U.S. Cl. CPC C12N 7/00; C12N 2710/10051; C12N 2760/16151

(57) ABSTRACT

The present invention relates to a method for virus capture or separation. More closely, the invention relates to a method for direct influenza and adenovirus capture using magnetic beads. The method allows direct separation from crude cell lysate in a rapid manner.

### FIELD OF THE INVENTION

The present invention relates to a method for virus capture or separation. More closely, the invention relates to a method for direct influenza and adenovirus capture using magnetic beads.

### BACKGROUND

There is an increasing demand for biopharmaceuticals such as viral vectors for gene therapy and monoclonal antibodies for immunotherapy. This arise from the discoveries of their capabilities in treatment of diseases such as cancer.

The traditional manufacturing process consists of an upstream, a midstream and a downstream part. Upstream is referring to cell expansion carrying target biomolecule until final harvesting, while downstream is processing the target molecule to acceptable purity and quality. Midstream is the interface between up- and downstream, and is aiming to remove bulk impurities from the harvesting and prepare the sample for column chromatography purification.

The adenovirus purification process consists of a capture step and polishing steps. The capture step is aiming to isolate the target molecule with high capacity, while polishing is aiming to remove residual impurities and achieve final high-level purity. The polishing step is flow through chromatography for both virus processes, and is aiming to capture the residual impurities, while the target virus is passing through the column without binding. The residual impurities in virus purification are mainly related to host cell proteins (HCP) and cell DNA remaining from upstream cell culture.

It is crucial that the solution is free from solid particles in conventional column chromatography due to the likelihood of blocking the column. This is the main objective for the midstream part of a manufacturing process. Solid impurities will clog the column and must thus be removed, for instance via filtration. However, filtration is a time-consuming and expensive process. Furthermore, influenza and adenovirus tend to aggregate and are unable to pass the filter pores, which causes extensive loss in virus yield.

Thus, there exists a need of capturing target molecules directly from a crude cell lysate suspension with expressed viruses with high selectivity, capacity and strong affinity, and successful enough to replace the traditional process using mid-stream filtration and column chromatography.

### SUMMARY OF THE INVENTION

The present invention provides a rapid and efficient method for virus purification comprising the following steps:
a) Addition of magnetic beads to a crude cell lysate suspension comprising target virus;
b) Homogenization and incubation of said suspension to allow binding of said target virus to ligands on said magnetic beads;
c) Capture of said magnetic beads;
d) Removal of supernatant from said magnetic beads;
e) Washing and repetition of steps c)-d); and
f) Addition of elution buffer, and collection of eluate containing target virus.

Preferably the magnetic beads are agarose beads with embedded 1-5 μm magnetite particles.

Preferably the magnetic beads have an average diameter of up to 120 μm, preferably up to 100 μm, such as 0-40 μm or 40-100 μm, or such as 0-37 μm or 37-100 μm. The magnetic beads comprise 2-6% agarose, preferably 3-5% agarose, most preferably 4% agarose.

In a preferred embodiment the magnetic beads comprise 4% agarose and have an average diameter of 0-37 μm.

The ligands are quaternary trimethylamine (Q) and/or sulfate(S), preferably at least the sulfate ligand is provided with a surface extender from the bead surface, such as a dextran extender.

When the target virus is adenovirus the ligands are Q-ligands and when the target virus is influenza virus the ligands are S-ligands.

The method of the invention presents several advantages compared to prior methods in that the binding of target virus to ligands may be performed directly from crude cell lysate and with binding of up to 90% of the target virus within a very short period of 30 minutes, such as within 15 minutes, or 5 minutes, or 1 minute.

### BRIEF DESCRIPTION OF THE DRAWINGS

FIG. 1 is a bar chart showing maximum capacities and dissociation constants of adenovirus binding for D×Q prototypes of the invention.

FIG. 2 is a graph showing adenovirus capture over time with D×Q prototypes of the invention.

FIG. 3 is a graph showing influenza binding capacities for D×S prototypes of the invention.

### DETAILED DESCRIPTION OF THE INVENTION

The invention will now be described more closely in association with the drawings and some non-limiting Examples. Novel ferrimagnetic chromatography resins for batch adsorption of adenovirus and influenza virus are provided that will increase the capacity and dissociation constant of the target molecule.

The invention relates to functionalization of MagSepharose prototypes (agarose beads with magnetite particles as described below) and methods of use for direct capture of adenovirus and influenza virus in batch adsorption mode. The beads are functionalized with a quaternary trimethylamine (Q) for adenovirus binding by anion-exchange, and dextran sulfate (D×S) for influenza virus binding by affinity capture.

The viruses used in the invention are H1N1 Influenza-A Virus Solomon Island/03/06 and Adenovirus type 5 Green Florescence Tagged Protein (AdV 5-GFP). When stating “Adenovirus” and “Influenza virus” in the below description, they refer to these specific viruses

Instead of mid-stream filtration the target molecule is captured directly from the cell lysate using batch adsorption, using magnetic beads with a specific affinity binding towards the target virus. The magnetic beads are highly selective and have high capacity.

The magnetic beads are captured by a magnet while carrying the target molecule. By separating the beads from the solution, soluble impurities are removed. The target molecule is eluted with a change of buffer composition (pH, salt) and collected.

The ferrimagnet magnetite (Fe₃O₄) is used in this invention. Magnetite has a spontaneous magnetization (as for all ferrimagnets), but reducing particle size of magnetite will result in “paramagnetic characteristics”, i.e. a small magnetic memory in the magnetite structure, but a good response to a magnetic field. The magnetite particles incorporated in the prototypes are 1-5 μm which leads to negligible magnetic memory and field. These properties will prevent the magnetic beads to permanently aggregate and affecting each other negatively in a batch adsorption purpose, but still have a good response to a magnetic field.

The magnetite particles are incorporated or embedded in agarose beads during the emulsification resulting in agarose beads with a magnetite core.

Both adenovirus and influenza virus are macromolecules (70-120 nm), and the beads must have pores large enough to allow diffusion to utilize the internal volume of the resin. However, the available material for modification and interaction is low in very porous material, which can lead to lower binding capacities. In the present invention this is solved using surface extenders, preferably dextran, a long chain polysaccharide. Dextran increases the available ligand coupling points and creates a three-dimensional structure for the target molecule to bind and may be coupled onto magnetite bead.

A trimethylamine (Q) ligand was selected for adenovirus binding. Dextran was first coupled onto the beads to increase the surface area for ligand coupling (and thus virus binding sites), and later functionalized with Q. This complex is abbreviated D×Q. The dextran itself does not have any binding properties toward adenovirus.

However, the dextran polymer has an active role for influenza virus binding, allowing multiple point of interaction with influenza virus. So, dextran sulfate can be viewed as a large ligand. The dextran sulfate complex is abbreviated D×S. Below the structure of the ligands D×Q (a), and D×S (b) are shown. The Q and S ligands are randomly coupled onto the dextran chain.

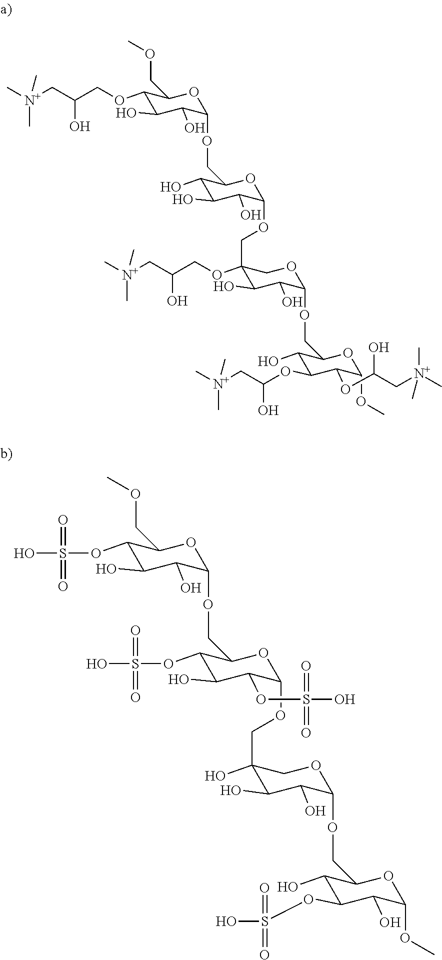

A preferred method of binding of virus molecules using magnetite beads is divided into following steps:
1) Add MagSepharose to the sample containing target molecule.
2) Homogenization and incubation.
3) Capture of MagSepharose by magnetic rack.
4) Removal of supernatant.
5) Addition of washing buffer, followed by step 2-4.
6) Optionally renew step 5 up to about five times.
7) Addition of elution buffer followed by step 2 and 3.
8) Collect the eluate containing target molecule.

### Prototypes

About 100 g of magnetite was encapsulated in 1.4 L of sedimented 4% and 2% agarose beads, for formation of the 2% and 4% MagSepharose resin (agarose resins with embedded 1-5 μm magnetite particles) used as starting material for production of the prototypes. The bead size is indicated after each protype of D×Q and D×S functionalized prototypes as described below.

For the porosity of 4% MagSepharose the Kd for a dextran of 110 KDa is 0.64 For the porosity of 2% MagSepharose the kD for a dextran of 110 Kda is about 0.8.

### 1. D×Q Functionalized MagSepharose

The D×Q functionalization was conducted on four different MagSepharose prototype resins:
1) 4% MagSepharose 0-37 μm,
2) 4% MagSepharose 37-100 μm,
3) 2% MagSepharose 0-37 μm, and
4) 2% MagSepharose 37-100 μm.

The following reactions were used to produce D×Q prototypes: epoxyactivation of gel and dextran coupling.

The amount of dextran coupled on the four base matrixes was determined by measuring the increase in dry weight, i.e. the weight of the dried resin before and after introduction of dextran. The values are summarized in Error! Reference source not found. As can be seen, values between 12-22 mg/mL were obtained.

TABLE 1

| | | Dextran Weight |
|---|---|---|
| LS Number^{a} | Base Matrix | [mg/mL gel] |
| LS-026104 | 4% MagSepharose 0-37 μm | 22 |
| LS-026194 | 4% MagSepharose 37-100 μm | 12 |
| LS-026380 | 2% MagSepharose 0-37 μm | N/A |
| LS-026252 | 2% MagSepharose 37-100 μm | 14 |

The dextran coupled resins were functionalized with glycidyltrimethylammonium chloride (GMAC). The hydroxyl groups on the dextran chains are reacting via a nucleophilic substitution with the epoxy function of the GMAC under basic conditions. The GMAC contains the desirable Q ligand, and the resin was functionalized as an anion-exchanger for adenovirus purification. See reaction schemes below.

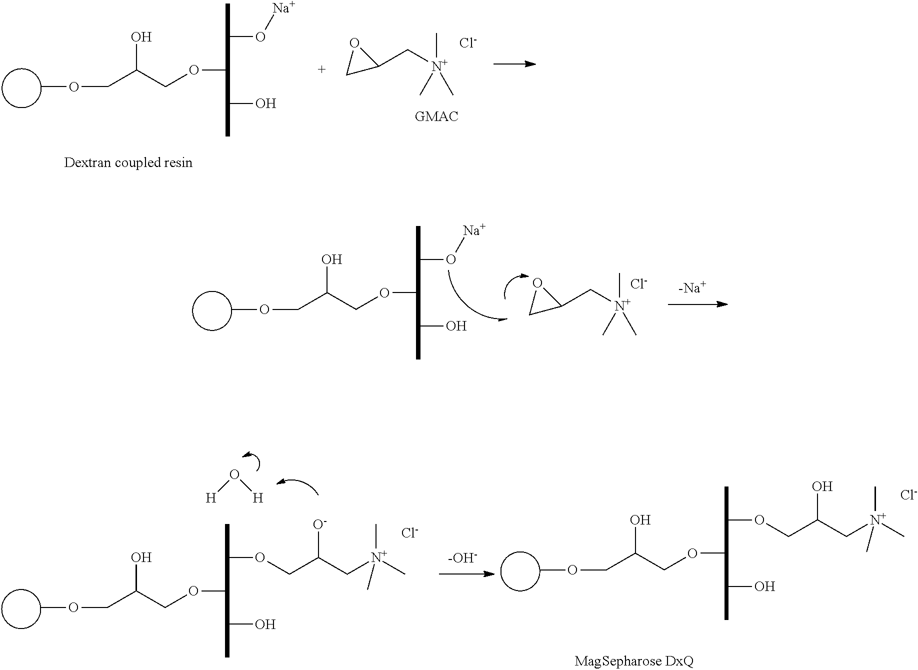

The ionic capacities obtained are shown in Error! Reference source not found. The 2% agarose based prototypes show a lower ligand density (53 and 92 μmol/mL gel) than the 4% agarose based prototypes (175 and 160 μmol/mL gel). This is probably due to lower agarose amount (i.e. less hydroxyl groups for attachment available) for the 2% prototypes. However, both prototypes are suitable to be candidates for anion-exchange for adenovirus application.

Presented in Error! Reference source not found. is also ion capacities based on “gram dry weight resin”. This was due to the difficulties of obtaining exactly 1 mL resin. However, no values could be obtained for the 2% prototypes because of limited available resin.

TABLE 2

| | | | Q Ion | |
|---|---|---|---|---|
| | | Dextran | Capacity | Q Ion Capacity |
| | | Weight [mg/ | [μmol/mL | [μmol per gram |
| LS Number | Base Matrix | mL gel] | gel] | dry weight gel] |
| LS-026104 | 4% MagSepharose 0-37 μm | 22 | 175 | 803 |
| LS-026194 | 4% MagSepharose 37-100 μm | 12 | 160 | 977 |
| LS-026380 | 2% MagSepharose 0-37 μm | N/A | 53 | N/A |
| LS-026252 | 2% MagSepharose 37-100 μm | 14 | 92 | N/A |

### 2. D×S Functionalized MagSepharose Beads

The D×S functionalization was conducted on three different agarose resins with embedded magnetite particles:
1) 4% MagSepharose 0-37 μm,
2) 4% MagSepharose 37-100 μm, and
3) 2% MagSepharose 0-100 μm.

To obtain two different particle size fractions of the 2% MagSepharose, the resin was sieved into two fractions: 2% MagSepharose D×S 0-37 μm and 2% MagSepharose D×S 37-100 μm before virus application test.

### Allylation

Introduction of an allyl group on the base matrix was conducted by a nucleophilic substitution with the epoxy function of AGE in a basic environment.

The allyl amount is presented in Error! Reference source not found, and shows higher allyl values for the 4% prototypes than the 2% as a probable result of higher agarose amount. The allyl amount in the unit “μmol per gram dry weight resin” is also presented in Error! Reference source not found, with absence of the value for the 4% MagSepharose 37 μm prototype due to machine error.

TABLE 3

| | | Allyl | Allyl Amount |
|---|---|---|---|
| LS | | Amount | [μmol per gram |
| Number | Base Matrix | [μmol/mL] | dry weight gel] |
| LS-026905 | 4% MagSepharose 0-37 μm | 214 | 1721 |
| LS-022420 | 4% MagSepharose 37-100 μm | 205 | N/A |
| LS-027121 | 2% MagSepharose 0-100 μm | 125 | 1242 |

### Bromination

The allyl group undergoes an allylic bromination by a bromine radical. The bromine radical reacts with the allylic hydrogen (i.e. the hydrogen at a carbon attached to a carbon-carbon double bond) in a one electron process, leaving one electron on the allylic carbon and forming an allylic radical with an equivalent resonance structure. The hydrogen is consequently substituted by a bromine at the allylic carbon. This intermediate is further reacting with water to generate an epoxide.

### Dextran Sulfate Coupling

The dextran sulfate chain was introduced to the epoxide groups of the resin by a nucleophilic attack in a basic environment as described above for the D×Q prototypes.

The dextran sulfate amount immobilized is presented in Error! Reference source not found. Due to volumetric sample preparation difficulties for the 2% prototype, only the ionic capacity in “μmol per gram dry weight resin” could be obtained.

TABLE 4

| | | Allyl | Allyl Amount | Dextran | Ion | Ionic capacity |
|---|---|---|---|---|---|---|
| | Base | Amount | [μmol per gram | Weight | capacity | [μmol per gram |
| LS Number | Matrix | [μmol/mL] | dry weight gel] | [mg/mL] | [μmol/mL] | dry weight gel] |
| LS-026905 | 4% | 214 | 1721 | 29 | 100 | 650 |
| | MagSepharose | | | | | |
| | 0-37 μm | | | | | |
| LS-022420 | 4% | 205 | N/A | 36 | 144 | N/A |
| | MagSepharose | | | | | |
| | 37-100 μm | | | | | |
| LS-027121 | 2% | 205 | N/A | N/A | N/A | 730 |
| | MagSepharose | | | | | |
| | 0-100 μm | | | | | |

Eight prototypes were successfully prepared, four with D×Q and four with D×S. These prototypes are presented in Error! Reference source not found . . .

TABLE 5

| Prototypes for Adenovirus | Prototypes for Influenza |
|---|---|
| Purification | Virus Purification |
| MagSepharose D × Q 4% agarose | MagSepharose D × S 4% agarose |
| Particle size: <37 μm | Particle size: <37 μm |
| MagSepharose D × Q 4% agarose | MagSepharose D × S 4% agarose |
| Particle size: 37-100 μm | Particle size: 37-100 μm |
| MagSepharose D × Q 2% agarose | MagSepharose D × S 2% agarose |
| Particle size: <37 μm | Particle size: <37 μm |
| MagSepharose D × Q 2% agarose | MagSepharose D × S 2% agarose |
| Particle size: 37-100 μm | Particle size: 37-100 μm |

Adenovirus binding with D×Q Prototypes

The adenovirus concentration of the start feed was determined to 6.40×10¹¹ virus particles per mL (VP/mL) using Quantitative Polymerase Chain reaction (qPCR). This feed was diluted ten times.

As a start, an adenovirus standard curve was set-up using different injection volumes of the start material onto anion exchange high performance liquid chromatography (AEX-HPLC) using analytical method. This method was used for a quick adenovirus titer determination in all samples.

The adenovirus binding was conducted as the process outlined below:

The experimental capacities (Q*) and adenovirus supernatant concentration after incubation (c*) for a specific volume of beads were determined. The experimental dissociation constant (K_{d}) and maximum capacity (Qₘₐₓ) can be estimated for each prototype using the Langmuir isotherm in the below equation.

The K_{d} and Qₘₐₓ for each prototype are shown in Error! Reference source not found. as a bar diagram. Note that a reference prototype labeled “Q MagSepharose 4FF” also was analyzed. It is a Q functionalized 4% MagSepharose with particle size distribution of 37-100 μm without dextran.

$$Q *= \frac{{Q}_{max} \cdot c *}{{K}_{d} + c *}$$

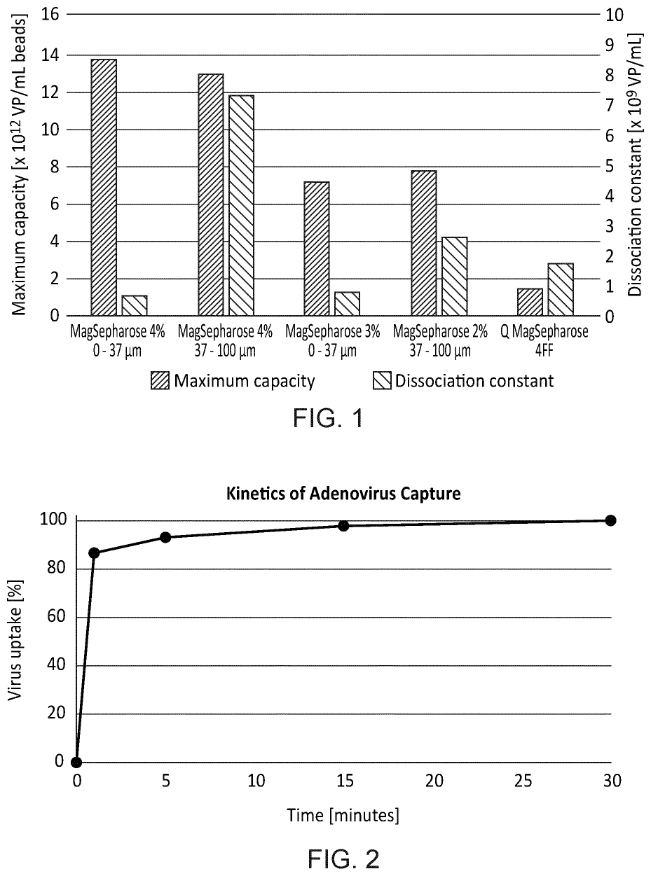

FIG. 1 shows that higher agarose content in the beads will result in higher capacity, and smaller beads will result in lower dissociation constants. Consequently, the 4% MagSepharose D×Q 0-37 μm prototype has the combination of highest capacity and lowest dissociation constant (Qₘₐₓ of 1.36×10¹³ VP per mL beads, and a K_{d} of 6.70×10⁸ VP per mL).

Furthermore, the use of dextran does result in a higher capacity regardless of particle size or percentage agarose, but does not clearly affect the dissociation constant. All new D×Q prototypes shows a high capacity for adenovirus purification. The incubation time in the experiment was one hour for adenovirus binding.

The Qₘₐₓ for the 4% MagSepharose D×Q 0-37 μm prototype was 1.36×10¹³ VP per mL beads, which means that only 2.98 μL beads would be needed to capture all the virus in a 1 mL feed with concentration of 4.05×10¹⁰ VP per mL (as for the experiment) if the dissociation constant is neglected.

A calculation of the experimental yield for the 4% MagSepharose D×Q 0-37 μm prototype compared to the 4% MagSepharose D×Q 37-100 μm prototype is presented in

Table 1. The prototypes have roughly the same Qₘₐₓ, but the higher dissociation constant for the 37-100 μm prototype reduces the yield from 99.3% to 92.5%.

$$c *= \frac{1}{2} [ {c}_{0} - {K}_{d} - \frac{{V}_{p} \cdot {Q}_{max}}{{V}_{batch}} + \sqrt{4 \cdot {c}_{0} \cdot {K}_{d} + {( \frac{{V}_{p} \cdot {Q}_{max}}{{V}_{batch}} - {c}_{0} + {K}_{d} )}^{2}} ]$$

TABLE 1
Overview of the impact of yield for different dissociation
constant but same capacities

| | 4% MagSepharose | 4% MagSepharose |
|---|---|---|
| | D × Q 0 −37 μm | D × Q 37 −100 μm |
| Cₒ [× 10¹⁰ VP/mL] | 4.05 | 4.05 |
| K_{d} [× 10⁸ VP/mL] | 6.70 | 73.0 |
| Vₚ [μL] | 10 | 10 |
| Qₘₐₓ [× 10¹³ VP/mL beads] | 1.36 | 1.28 |
| V_{feed} [L] | 1000 | 1000 |
| c* (by Eq. Error! Reference | 2.81 × 108 | 3.02 × 109 |
| source not found.) [VP/mL] | | |
| $Yield ( 1 - \frac{c *}{{c}_{0}} ) [ \% ]$ | 99.3 | 92.5 |

A binding kinetic study was performed for testing how fast the adenovirus was captured by the 4% MagSepharose D×Q 0-37 μm prototype. The unbound virus content was determined by taking supernatant samples at different times during incubation, followed by a HPLC analysis. Samples were taken after incubation times of: t= [1 min, 5 min, 15 min, 30 min]. The loading of beads was optimized to obtain a 100% yield (15 μL beads for 1 mL adenovirus feed with 4.05×10¹⁰ VP/mL). See Error! Reference source not found. for percent virus uptake after time t.

FIG. 2 shows that all adenovirus was captured after thirty minutes incubation, and 86.7% is already captured after one minute. This indicates that most of the adenovirus binding occurs under the first minute.

### Influenza Virus Binding by D×S Prototypes

D×S MagSepharose prototypes were tested for ability to capture the influenza virus directly from the cell lysate without competition from other host cell fragments. The influenza virus application test was conducted as outlined below.

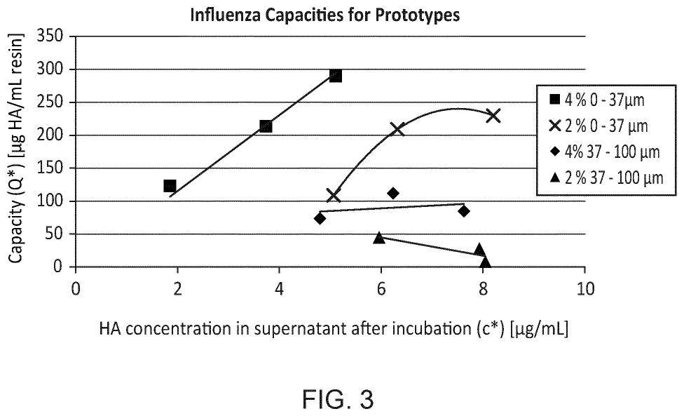

The influenza amount was determined by the measurement of the influenza surface protein hemagglutinin (HA) in the samples. The experimental capacities (Q*) and the HA supernatant concentrations after incubation (c*) for a specific volume of beads are shown in FIG. 3.

FIG. 3 shows that the prototypes with smaller bead sizes (<37 μm) showed higher binding capacities than the corresponding 37-100 μm. Comparing the <37 μm prototypes, the 4% has lower dissociation constant than the 2% (the 4% prototype shifted more to the left, i.e. less HA in supernatant after incubation).

The 4% MagSepharose D×S 0-37 μm was the best candidate for influenza virus, showing highest binding capacity and lowest dissociation constant.

The main advantage of using functionalized magnetite resins was clearly shown for influenza virus, allowing to omit the time consuming midstream filtration step and retaining a comparable overall virus yield.

### EXPERIMENTAL PART

### Experiment 1: Preparation of D×Q Prototype

### Epoxy Activation

Drained MagSepharose (unfunctionalized agarose beads with embedded 1-5 μm magnetite particles) was washed with DW 10×1 GV in a SF P3. The resin was transferred to a 250 mL 3N-RBF flask with DW, and placed into a 27° C. water bath, and fitted with an overhead stirrer. Stirring applied at ˜300 rpm until a homogenous slurry was obtained. NaOH pellets were added to the slurry, and stirring applied for fifteen more minutes. ECH was added, and the reaction left for two hours with stirring. The resin was washed with DW in SF P3 to neutral pH (measured by pH stick) and then vacuum drained and the weight recorded.

### Dextran Coupling

D×T 40 and DW were added to a 250 mL 3N-RBF flask, and overhead stirred for at least four hours at low rpm (80 rpm).

Epoxy activated resin was added directly to the dissolved dextran in the 3N-RBF. The flask was placed in a 40° C. water bath under overhead stirring at low rate (˜120 rpm). A reflux condenser was used for minimizing evaporation. DW was added to make the slurry less viscous. Nitrogen gas was bubbled in the solution to drive away oxygen for twenty minutes. 50% NaOH and sodium borohydride (NaBH₄) were added and the reaction was left for 18 hours, after which DW and 60% acetic acid (HAc) were added to stop the reaction. The resin was washed to neutral pH on a SF P3.

### GMAC Coupling

Dextran coupled resin was washed with 2 M NaOH 3×1 GV in SF P3, drained and weight recorded. The resin was transferred to a 250 mL 3N-RBF, to which GMAC was added. Coupling was performed for 18 h at room temperature with overhead stirring at ˜300 rpm.

The resin was washed with DW in SF P3 until neutral pH (measured using pH strip).) Resin was drained and stored in the fridge in a 20% EtOH at 65% slurry concentration.

D×Q 4% MagSepharose Particle Size 0-37 μm

### Step 1-Epoxy Activation

112.5 g of 4% MagSepharose 0-37 μm and 33.6 mL DW was treated with 10.9 g NaOH and 21.3 mL ECH. Giving 109.9 g of resin.

Dry weight measurements were performed in triplicates giving an average of 98 mg per mL resin.

### Step 2-Dextran Coupling

26 mL DW and 41 g D×T 40 for dextran solving. 90 g epoxy activated resin added with 3 mL DW, followed by 4.9 mL 50% NaOH and 0.19 g NaBH₄.

Reaction stopped by adding 40 mL DW and 15 mL 60% HAc.

Dry weight measurements were performed in triplicates giving an average of 120 mg per mL resin.

### Step 3-GMAC Coupling:

Drained resin weight to 89.5 g, and 10⁸ mL GMAC added. After reaction: washed and drained resin weight to 77.8 g, whereof 66.2 g resin stored.

Volumetric ion capacity titrations were performed in triplicates giving an average of 175 μmol per mL resin. Ion capacity titrations on weight were performed in duplicates with an average of 803 μmol per gram dry weight resin.

D×Q 4% MagSepharose Particle Size 37-100 μm

Step 1-Epoxy Activation 112.7 g of 4% MagSepharose 37-100 μm and 33.6 mL DW was treated with 10.9 g NaOH and 21.3 mL ECH. Giving 10⁸.9 g of resin.

Dry weight measurements were performed in duplicates giving an average of 117 mg per mL resin.

### Step 2-Dextran Coupling

22 mL DW and 38.9 g D×T 40 for dextran solving. 90 g epoxy activated resin added with 3 mL DW, followed by 4.9 mL 50% NaOH and 0.194 g NaBH₄.

Reaction stopped by adding 40 mL DW and 15 mL 60% HAc.

Dry weight measurements were performed in duplicates giving an average of 129 mg per mL resin.

### Step 3-GMAC Coupling:

Drained resin weight to 85.41 g, and 103 mL GMAC added. After reaction: washed and drained resin weight to 73.7 g, whereof 59.6 g resin stored.

Volumetric ion capacity titrations were performed in duplicates giving an average of 160 μmol per mL resin. Ion capacity titrations on weight were performed in duplicates with an average of 977 μmol per gram dry weight resin.

D×Q 2% MagSepharose Particle Size 0-37 μm

### Step 1-Epoxy Activation

22 g of 2% MagSepharose 0-37 μm and 6.6 mL DW was treated with 2.1 g NaOH and 4.2 mL ECH. Giving 21 g of resin.

Dry weight measurements were not performed because of limited available resin.

### Step 2-Dextran Coupling

6 mL DW and 10.8 g D×T 40 for dextran solving. 15 g epoxy activated resin added with 1 mL DW, followed by 0.80 mL 50% NaOH and 0.030 g NaBH₄. Note: weight Dextran was for 25 g resin, but only 15 g was used.

Reaction stopped by adding 20 mL DW and 10 mL 60% HAc.

Dry weight measurements were not performed because of limited available resin.

### Step 3-GMAC Coupling:

Drained resin weight to 10.2 g, and 12.2 mL GMAC added. After reaction: washed and drained resin weight to 10.6 g, whereof 7.5 g resin stored.

Volumetric ion capacity titration conducted in duplicates with average of 53 μmol per mL resin. No ion capacity titration on weight conducted due to limited available resin.

D×Q 2% MagSepharose Particle Size 37-100 μm

### Step 1-Epoxy Activation

57.7 g 2% MagSepharose 37-100 μm and 17.3 mL DW was treated with 5.7 g NaOH and 11 mL ECH. Giving 51.0 g of resin.

Dry weight measurements were performed in triplicates giving an average of 94 mg per mL resin.

### Step 2-Dextran Coupling

110 mL DW and 26.1 g D×T 40 for dextran solving. 48 g epoxy activated resin added with 1 mL DW, followed by 2.6 mL 50% NaOH and 0.10 g NaBH₄. Note: weight Dextran was for 60 g resin, but 48 g was used.

Reaction stopped by adding 40 mL DW and 13 mL 60% HAc.

Dry weight measurements were performed in duplicates giving an average of 10⁸ mg per mL resin.

### Step 3-GMAC Coupling:

Drained resin weight to 50.5 g, and 61 mL GMAC added. After reaction: washed and drained resin weight to 47.1 g, whereof 44.0 g resin stored.

Volumetric ion capacity titrations were performed in duplicates giving an average of 92 μmol per mL resin. No ion capacity titration on weight conducted due to limited available resin.

### Experiment 2: Preparation of D×S Prototype

### Allylation

Drained MagSepharose (unfunctionalized agarose beads with embedded 1-5 μm magnetite particles) was washed with DW 10×1 GV in a SF P3, and drained weight recorded. The resin was transferred to a 250 mL 3N-RBF with an equal amount of 50% NaOH. Stirring occurred (˜150 rpm) in 50° C. water bath for thirty minutes. A reflux condenser was used for minimizing evaporation. AGE was then added, and the reaction left under stirring (˜300 rpm) for 18 h.

The resin was washed with DW 10×1 GV in SF P3, vacuum drained and the weight measured.

### Bromination

The activated allyl resin was transferred to a 250 mL 3N-RBF along with DW and sodium acetate trihydrate (C₂H₉NaO₅). The slurry was stirred (˜150 rpm, room temperature) for 40 minutes. Bromine was added and the stirring speed increased to “500 rpm for 15 minutes. Sodium formate (HCOONa) was added portion wise under the high stirring, and additionally stirred for 15 minutes. The resin was then washed with DW 10×1 GV in a SF P3, and drained weight recorded.

### Dextran Sulfate Coupling

The brominated resin was transferred to a 250 mL 3N-RBF with DW, creating a 50-50 slurry under stirring in low rate (˜150 rpm, room temperature). D×S-AB was added, and the reaction was left under stirring for 1 h. The flask was immersed in a 33° C. water bath, 50% NaOH was added and the mixture left under stirring for 18 h. The coupling was stopped by adding DW, and the resin washed with DW 10×1 GV in a SF P3. Resin was drained and stored in the fridge in a 20% EtOH at 65% slurry concentration.

D×S 4% MagSepharose Particle Size 0-37 μm

### Step 1-Allylation.

Drained weight of 41.6 g was treated with 41.6 mL 50% NaOH and 10 mL AGE. Giving 35 g resin. Dry weight measurements were performed in duplicates giving an average of 125 mg per mL resin. Volumetric allyl titration and allyl titration on weight were performed in duplicates giving an average of 214 μmol per mL resin and 1721 μmol per gram dry weight resin, respectively.

### Step 2-Bromination.

35 mL DW, 1.25 g sodium acetate trihydrate, 1 mL bromine and 1.8 sodium formate were added for bromination. Giving 33 g resin.

### Step 3-Dextran Sulfate Coupling.

62 mL DW, 33 g D×S-AB and 3 mL 50% NaOH were added for dextran sulfate coupling. Reaction stopped by adding 100 mL DW, and washed and drained resin weight to 27 g, whereof 20 g resin stored.

Dry weight measurements were performed in duplicates giving an average of 153 mg per mL resin. Volumetric ion capacity titration and ion capacity titration on weight were performed in duplicates giving an average of 100 μmol per mL resin and 650 μmol per gram dry weight resin, respectively.

### D×S 2% Magsepharose Particle Size 0-100 μm

### Step 1-Mixing Two Fractions of 2% MagSepharose Resin.

Two fractions were mixed to a homogenous slurry in a 250 mL 3N-RBF containing 45 mL DW: 15 mL 2% MagSepharose 0-37 μm and 30 mL 2% MagSepharose 37-100 μm. This resin was washed with DW 10×1 GV in SF P3, and drained weight to 44 g.

### Step 2-Allylation.

Drained weight of 44 g was treated with 45 mL 50% NaOH and 10 mL AGE. Giving 38 g resin. Dry weight measurements were performed in duplicates giving an average of 100 mg per mL resin. Volumetric allyl titration and allyl titration on weight were performed in duplicates giving an average of 125 μmol per mL resin and 1242 μmol per gram dry weight resin, respectively.

### Step 3-Bromination:

35 mL DW, 1.37 g sodium acetate trihydrate, 1 mL bromine and 1.8 sodium formate were added for bromination. Giving 35 g resin.

### Step 4-Dextran Sulfate Coupling.

45 mL DW, 33 g D×S-AB and 3 mL 50% NaOH were added for dextran sulfate coupling. Reaction stopped by adding 100 mL DW. Instead of washing in SF P3, a MagRack suitable for 500 mL Duran™ flask was used. The washing procedure was the same as shown in Error! Reference source not found. Resin drained in SF P3, and weight to 33 g. Note: the resin was not stored before sieving. Dry weight measurements were not performed due to complications with volumetric cubing. No volumetric ion capacity titration could either be performed. Ion capacity titration on weight were performed in duplicates giving an average of 730 μmol per gram dry weight resin.

### Step 6-Sieving.

The resin above was sieved on 37 μm on Sweco™ Separator (model no. S18S) and sieving cloth 37° C. (18A8A, M6370, 37 μm), getting two fractions: 0-37 μm and 37-100 μm. After sedimentation, the fraction 0-37 μm had a volume of about 9 mL, and the one of 37-100 μm about 28 mL.

### Experiment 3: Adenovirus Binding of D×Q Prototype

The stored D×Q functionalized prototypes as well as the reference prototype Q MagSepharose 4FF were all pipetted by volumes of 10, 7, 5 and 2 μL to individual 2 mL Eppendorf® tubes. 1 mL of 20 mM Tris+300 mM NaCl pH 8.0 was added to each Eppendorf tube for washing, and each sample was washed using MagRack 6. Washing repeated three cycles, and the last volume of supernatant waste at the bottom of the Eppendorf tubes were pipetted away.

1 mL adenovirus feed (AdV 5-GFP; concentration: 4.05×10¹⁰ VP/mL) was pipetted to each Eppendorf tube. Incubation were conducted for one hour using a shaking table (300 rpm).

The incubated beads were all trapped using MagRack 6, and 500 μL supernatant samples were pipetted to individual 2 mL HPLC vials (Agilent Technologies) when beads were still trapped. The HPLC vials were placed in an Agilent Technologies 1260 Infinity Bioinert system for chromatographic analysis of adenovirus content. Following chromatographic settings were used for the analysis.

| UV-detection: | 260 | nm |
|---|---|---|
| Injection volume: | 100 | μL |
| Sample temperature: | 8° | C. |
| A-buffer: | 20 mM Tris pH 7.5 | |
| B-buffer: | 20 mM Tris + 1M NaCl pH 7.5 | |
| Flow-rate: | 1.5 | mL/min |

| | |
|---|---|
| Column: | Q Sepharose XL ™ 1.0 mL in Tricorn ™ 5 column |
| Gradient: | 100% A-buffer/0% B-buffer: 3 min |
| | 30% A-buffer/70% B-buffer: 3-13 min |
| | 30% A-buffer/70% B-buffer: 13-14 min |
| | 0% A-buffer/100% B-buffer: 14-16 min |
| | 0% A-buffer/100% B-buffer: 16-23 min |

Following peak areas were obtained for the supernatant samples, Table 7.

TABLE 7

| Volume | Peak Area [mAUs] | | | | |
|---|---|---|---|---|---|
| Beads | 4% MagSepharose | 4% MagSepharose | 2% MagSepharose | 2% MagSepharose | Q MagSepharose |
| [μL] | D × Q 0-37 μm | D × Q 37-100 μm | D × Q 0-37 μm | D × Q 37-100 μm | 4FF |
| 10 | 0.37 | 11.1 | 3.4 | 11.3 | 7.6 |
| 7 | 2.65 | 24.8 | 30.5 | 19.9 | 23.1 |
| 5 | 4.42 | 32.3 | 32.6 | 48.1 | 49.4 |
| 2 | 61 | 93.1 | 107.1 | 114.3 | 49.9 |

### Experiment 4: Influenza Virus Binding on D×S Prototypes

From the D×S functionalized prototypes were volumes of 50, 100, 200 and 500 μL beads pipetted into individual 10 mL Falcon tubes. 10 mL 20 mM Tris pH 7.5+150 mM NaCl was added for washing in total three times. The last volume of supernatant waste at the bottom of the Falcon tubes were pipetted away.

10 mL crude influenza feed (H1N1 Influenza-A Virus Solomon Island/03/06) was pipetted into each Falcon tube, and incubation was conducted for one hour using head-over-head rotation (˜60 rpm).

All beads in the sample tubes were individually trapped by MagRack Maxi after incubation, and 2 mL supernatant samples were saved in individual 2 mL Eppendorf tubes for HA analysis. The remaining incubation supernatants were decanted to waste.

The beads were washed three times with 10 mL 20 mM Tris pH 7.5+150 mM NaCl in MagRack Maxi. The last volumes of supernatant wastes at the bottom of the Falcon tubes were pipetted away. Elution of influenza virus was conducted by adding 1 mL 20 mM Tris pH 7.5+0.75 M NaCl to each system, and stirring the Falcon tubes for ten minutes by hand. Beads were then captured by

MagRack Maxi, and 2 mL elution sample from each system were saved in separate 2 mL Eppendorf tubes. The remaining elution supernatant was decanted to waste.

This procedure was repeated in total three times, and gave three elution samples for analysis.

### Results for D×Q Prototypes

The experimental results for direct capture of adenovirus using D×Q prototypes are shown in this section. See Tables 8-13 below.

| | |
|---|---|
| Volume Beads | Volume beads for virus incubation. |
| Volume Feed | The volume adenovirus feed used for |
| | incubation. |
| Titer Feed | The titer of adenovirus feed used for |
| | incubation (denoted c₀ in the relevant equation). |
| Peak Area | The chromatograph peak area of the supernatant |
| | sample, i.e. the virus in mobile phase not |
| | captured by the beads at equilibrium. |
| Virus Titer in | The chromatogram area's corresponding virus |
| Supernatant After | amount using the relevant equation. |
| Incubation (c*) | |
| Virus Bound (Q*) | The specific capacity for a volume of beads. |
| | Calculated by deducting the captured beads |
| | from the start titer feed and divide by volume |
| | beads (unit in VP per mL resin). |

TABLE 8
Adenovirus c* and Q* for D × Q MagSepharose 4% 0-37 μm

| | | Titer | | Virus Titer in | |
|---|---|---|---|---|---|
| Volume | Volume | Feed | Peak | Supernatant After | Virus Bound |
| Beads | Feed | [×10¹⁰ | Area | Incubation (c*) | (Q*) [×10¹² |
| [μL] | [μL] | VP/mL] | [mAUs] | [×10⁹ VP/mL] | VP/mL resin] |
| 10 | 1000 | 4.05 | 0.37 | 0.0844 | 4.04 |
| 7 | 1000 | 4.05 | 2.65 | 0.604 | 5.70 |
| 5 | 1000 | 4.05 | 4.42 | 1.01 | 7.89 |
| 2 | 1000 | 4.05 | 61 | 13.9 | 13.3 |

TABLE 9
Adenovirus c* and Q* for D × Q MagSepharose 4% 37-100 μm

| | | Titer | | Virus Titer in | |
|---|---|---|---|---|---|
| Volume | Volume | Feed | Peak | Supernatant After | Virus Bound |
| Beads | Feed | [×10¹⁰ | Area | Incubation (c*) | (Q*) [×10¹² |
| [μL] | [μL] | VP/mL] | [mAUs] | [×10⁹ VP/mL] | VP/mL resin] |
| 10 | 1000 | 4.05 | 11.1 | 2.53 | 3.79 |
| 7 | 1000 | 4.05 | 24.8 | 5.65 | 4.97 |
| 5 | 1000 | 4.05 | 32.3 | 7.36 | 6.62 |
| 2 | 1000 | 4.05 | 93.1 | 21.2 | 9.62 |

TABLE 10
Adenovirus c* and Q* for D × Q MagSepharose 2% 0-37 μm

| | | Titer | | Virus Titer in | |
|---|---|---|---|---|---|
| Volume | Volume | Feed | Peak | Supernatant After | Virus Bound |
| Beads | Feed | [×10¹⁰ | Area | Incubation (c*) | (Q*) [×10¹² |
| [μL] | [μL] | VP/mL] | [mAUs] | [×10⁹ VP/mL] | VP/mL resin] |
| 10 | 1000 | 4.05 | 3.44 | 0.784 | 3.97 |
| 7 | 1000 | 4.05 | 30.5 | 6.95 | 4.79 |
| 5 | 1000 | 4.05 | 32.6 | 7.43 | 6.61 |
| 2 | 1000 | 4.05 | 107.1 | 24.4 | 8.03 |

TABLE 11
Adenovirus c* and Q* for D × Q MagSepharose 2% 37-100 μm

| | | Titer | | Virus Titer in | |
|---|---|---|---|---|---|
| Volume | Volume | Feed | Peak | Supernatant After | Virus Bound |
| Beads | Feed | [×10¹⁰ | Area | Incubation (c*) | (Q*) [×10¹² |
| [μL] | [μL] | VP/mL] | [mAUs] | [×10⁹ VP/mL] | VP/mL resin] |
| 10 | 1000 | 4.05 | 11.25 | 2.56 | 3.79 |
| 7 | 1000 | 4.05 | 19.85 | 4.53 | 5.13 |
| 5 | 1000 | 4.05 | 48.05 | 11.0 | 5.90 |
| 2 | 1000 | 4.05 | 114.3 | 26.1 | 7.20 |

TABLE 12
Adenovirus c* and Q* for Q MagSepharose 4FF

| | | Titer | | Virus Titer in | |
|---|---|---|---|---|---|
| Volume | Volume | Feed | Peak | Supernatant After | Virus Bound |
| Beads | Feed | [×10¹⁰ | Area | Incubation (c*) | (Q*) [×10¹² |
| [μL] | [μL] | VP/mL] | [mAUs] | [×10⁹ VP/mL] | VP/mL resin] |
| 50 | 1000 | 6.34 | 33.3 | 7.59 | 1.12 |
| 30 | 1000 | 6.34 | 101.4 | 23.1 | 1.34 |
| 10 | 1000 | 6.34 | 216.7 | 49.4 | 1.40 |
| 5 | 1000 | 6.34 | 218.8 | 49.9 | 2.70 |

TABLE 13

| | Maximum Capacity | |
|---|---|---|
| | Using Langmuir | Dissociation Constant |
| | Isotherm (Qₘₐₓ) | Using Langmuir |
| | [×10¹² VP/ | Isotherm (K_{d}) |
| Prototype | mL beads] | [×10⁸ VP/mL] |
| 4% MagSepharose D × Q | 13.6 | 6.70 |
| 0-37 μm | | |
| 4% MagSepharose D × Q | 12.8 | 73.0 |
| 37-100 μm | | |
| 2% MagSepharose D × Q | 7.11 | 7.73 |
| 0-37 μm | | |
| 2% MagSepharose D × Q | 7.72 | 25.9 |
| 37-100 μm | | |
| Q MagSepharose 4FF | 1.46 | 17.5 |
| 37-100 μm | | |

### Adenovirus Binding Kinetic Study

Table 14 below presents the data for percentage virus uptake as a function of time.

| | |
|---|---|
| Peak area start feed: | 167.9 mAUs |
| (Injection volume: 100 uL; virus | |
| feed conc. 4.05 ×10¹⁰ VP/mL) | |

| Time [min] | Peak Area [mAUs] | Percent Virus Uptake |
|---|---|---|
| 0 | 0 | 0.0% |
| 1 | 22.4 | 86.7% |
| 5 | 11.6 | 93.1% |
| 15 | 3.7 | 97.8% |
| 30 | 0 | 100% |

### Results for D×S Prototypes

The experimental results for direct capture of influenza virus using D×S prototypes are shown in this section, see Tables 14-17 below.

| | |
|---|---|
| Volume beads | Volume beads for virus incubation. |
| HA concentration in | The HA concentration in the supernatant after incubation. |
| supernatant after | |
| incubation (c*) | |
| HA content in | The HA content in the supernatant after incubation. |
| supernatant after | (c*x Start feed volume) |
| incubation | |
| HA bound by beads | The calculated captured HA from the supernatant data. |
| | (Start HA content-HA content in supernatant after incubation) |
| Capacity (Q*) | The specific capacity for a volume of beads. |
| | $( \frac{HA bound by beads}{Volume beads} ) .$ |
| Recovered HA after | The total μg recovered HA after three elution steps. |
| elution | Analyzed in Biacore. |
| Step yield | How much HA eluted compared to bound. |
| | $( 1 - \frac{Recovered HA after elution}{HA bound by beads} )$ |
| Overall HA yield | How much HA eluted compared to initial HA provided. |
| | $That is : 1 - \frac{Recovered HA after elution}{Start HA content} .$ |

TABLE 14
Influenza virus c*, Q* and yields for DxS 4% MagSepharose 0-37 μm

| | Start feed concentration: | | 8.008 μg HA per mL feed (μg HA/mL) | | | | |
|---|---|---|---|---|---|---|---|
| | Start feed volume: | | 10 mL | | | | |
| | Start HA content: | | 8.008 μg HA/mL × 10 mL = 80.08 μg HA | | | | |
| | HA concentration | HA content in | | Capacity | Recovered | | Overall |
| Volume | in supernatant | supernatant | HA bound | (Q*) | HA after | Step | HA |
| beads | after incubation | after incubation | by beads | [μg/mL | elution | yield | yield |
| [μL] | (c*) [μg/mL] | [μg] | [μg] | beads] | [μg] | [%] | [%] |
| 500 | 1.853 | 18.53 | 61.55 | 123.1 | 44.398 | 72.1 | 55.44 |
| 200 | 3.738 | 37.38 | 42.7 | 213.5 | 33.945 | 79.5 | 42.39 |
| 100 | 5.109 | 51.09 | 28.99 | 289.9 | 24.803 | 85.6 | 30.97 |

TABLE 15
Influenza virus c*, Q* and yields for DxS 4% MagSepharose 37-100 μm

| | |
|---|---|
| Start feed concentration: | 8.470 μg HA/mL |
| Start feed volume: | 10 mL |
| Start HA content: | 84.70 μg HA |

| | HA concentration | HA content in | | Capacity | Recovered | | Overall |
|---|---|---|---|---|---|---|---|
| Volume | in supernatant | supernatant | HA bound | (Q*) | HA after | Step | HA |
| beads | after incubation | after incubation | by beads | [μg/mL | elution | yield | yield |
| [μL] | (c*) [μg/mL] | [μg] | [μg] | beads] | [μg] | [%] | [%] |
| 500 | 4.795 | 47.95 | 36.75 | 73.5 | 32.644 | 88.8 | 38.54 |
| 200 | 6.235 | 62.35 | 22.35 | 111.75 | 20.74 | 92.8 | 24.49 |
| 100 | 7.624 | 76.24 | 8.46 | 84.6 | 7.11 | 84.0 | 8.39 |

TABLE 16
Influenza virus c*, Q* and yields for DxS 2% MagSepharose 0-37 μm

| | |
|---|---|
| Start feed concentration: | 10.50 μg HA/mL |
| Start feed volume: | 10 mL |
| Start HA content: | 105.0 μg HA |

| | HA concentration | HA content in | | Capacity | Recovered | | Overall |
|---|---|---|---|---|---|---|---|
| Volume | in supernatant | supernatant | HA bound | (Q*) | HA after | Step | HA |
| beads | after incubation | after incubation | by beads | [μg/mL | elution | yield | yield |
| [μL] | (c*) [μg/mL] | [μg] | [μg] | beads] | [μg] | [%] | [%] |
| 500 | 5.065 | 50.65 | 54.35 | 108.7 | 34.938 | 64.3 | 33.27 |
| 200 | 6.32 | 63.2 | 41.8 | 209 | 26.212 | 62.7 | 24.96 |
| 100 | 8.204 | 82.04 | 22.96 | 229.6 | 10.961 | 47.7 | 10.44 |

TABLE 17
Influenza virus c*, Q* and yields for DxS 2% MagSepharose 37-100 μm

| | |
|---|---|
| Start feed concentration: | 8.200 μg HA/mL |
| Start feed volume: | 10 mL |
| Start HA content: | 82.00 μg HA |

| | HA concentration | HA content in | | Capacity | Recovered | | Overall |
|---|---|---|---|---|---|---|---|
| Volume | in supernatant | supernatant | HA bound | (Q*) | HA after | Step | HA |
| beads | after incubation | after incubation | by beads | [μg/mL | elution | yield | yield |
| [μL] | (c*) [μg/mL] | [μg] | [μg] | beads] | [μg] | [%] | [%] |
| 500 | 5.959 | 59.59 | 22.41 | 44.82 | 20.422 | 91.1 | 24.90 |
| 200 | 8.045 | 80.45 | 1.55 | 7.75 | 6.486 | 418.5 | 7.91 |
| 100 | 7.926 | 79.26 | 2.74 | 27.4 | 1.368 | 49.9 | 1.67 |

1. A method for virus purification comprising the following steps:
a) Addition of magnetic beads to a crude cell lysate suspension comprising target virus;
b) Homogenization and incubation of said suspension to allow binding of said target virus to ligands on said magnetic beads;
c) Capture of said magnetic beads;
d) Removal of supernatant from said magnetic beads;
e) Washing and repetition of steps c)-d); and
f) Addition of elution buffer, and collection of eluate containing target virus.

2. Method according to claim 1, wherein the magnetic beads are agarose beads with embedded 1-5 μm magnetite particles.

3. Method according to claim 1 or 2, wherein the magnetic beads have an average diameter of up to 120 μm, preferably up to 100 μm, such as 0-40 μm or 40-100 μm, or such as 0-37 μm or 37-100 μm.

4. Method according to claim 1, 2 or 3, wherein the magnetic beads comprise 2-6% agarose, preferably 3-5% agarose, most preferably 4% agarose.

5. Method according to one or more of the above claims, wherein said ligands are quaternary trimethylamine (Q) and/or dextran sulfate (D×S).

6. Method according to claim 5, wherein the ligands are provided on dextran extenders.

7. Method according to one or more of the above claims, wherein the target virus is adenovirus and the ligands are Q-ligands.

8. Method according to one or more of the above claims, wherein the target virus is influenza virus and the ligands are S-ligands.

9. Method according to one or more of the above claims, wherein said binding of target virus to ligands occurs within 30 minutes, such as within 15 minutes, or 5 minutes, or 1 minute.

10. Method according to one or more of the above claims, wherein the magnetic beads comprise 4% agarose and have an average diameter of 0-37 μm.

11. Method according to one or more of the above claims, wherein step e) is repeated up to five times
